(19) United States
(12) Patent Application Publication
(10) Pub. No.: US 2006/0004264 A1
(43) Pub. Date: Jan. 5, 2006

(54) Medical device data management

(76) Inventors: Robert W. Rudowski II, Mattawan, MI (US); Ronald C. Colcernian, Waterford, MI (US)

Correspondence Address: CARLSON, GASKEY & OLDS, P.C., 400 WEST MAPLE ROAD, SUITE 350, BIRMINGHAM, MI, 48009, US

(21) Appl. No.: 11/112,862

(22) Filed: Apr. 22, 2005

Related U.S. Application Data
(60) Provisional application No. 60/564,331, filed on Apr. 22, 2004.

(51) Int. Cl. A61B 5/00

(52) U.S. Cl. 600/300

(57) ABSTRACT

A system for handling data from medical testing devices ensures the integrity of the data to facilitate accurate handling of the data and to facilitate payment for legitimately needed therapy or medical equipment. A disclosed example includes a communication device that is useful for extracting data from a testing device in a secure manner. In one example, the data is encrypted when extracted by the communication device. Communications with a control center are also protected. The control center automatically verifies whether the contents of the extracted data fit within expected criteria.

This application claims priority to U.S. Provisional Patent Application No. 60/564,331, filed Apr. 22, 2004.

### BACKGROUND OF THE INVENTION

1. Field of the Invention

This invention generally relates to managing data collected from devices useful in the medical field. More particularly, this invention relates to ensuring the integrity of such data, in part, for assisting in legitimate payment for medical services.

2. Description of the Related Art

There are a variety of uses for medically related devices that gather diagnostic data regarding a patient. One example device is a pulse oximeter that operates in a generally known manner for monitoring an individual's pulse rate and blood oxygen level. A pulse oximeter records two values regarding an individual's pulse and oxygen level. In one example, such readings or measurements are taken every four seconds. Some devices include the ability to record such data on an every-second basis.

Pulse oximeters are often used in hospital settings to provide information to medical professionals caring for a patient. Pulse oximeters are also used for purposes of determining whether an individual qualifies for in home medical services such as oxygen therapy. In the latter case, the pulse oximeter data provides data indicative of whether the individual patient will benefit from oxygen therapy and whether their current health status warrants such therapy.

Depending on whether the testing or assessment will take place in a patient's home or a testing facility, various steps must be completed. At a testing facility, for example, arrangements must be made to accommodate the patient and complete the necessary data accumulation using appropriate testing equipment. Once the information is extracted from the testing equipment, it then has to be prepared for analysis. In the in-home testing situation, a therapist must take the testing device to the patient's home where the test will be performed. In many situations, the test will have a relatively lengthy duration such that it does not make sense for the therapist to remain at the patient's home. In such situations, the therapist must return to the patient's home after the test is completed to finish the process and then eventually bring the testing device or equipment to the appropriate provider facility so that the data from the test equipment can be obtained by the provider.

The results of the test obtained from the device are then put into a useable format and typically faxed to the physician's office. After receiving the fax, the physician is able to evaluate the test results and then issue an order or prescription when appropriate. This information must then be conveyed to a home medical equipment provider, by facsimile for example. At this point, the home medical equipment provider is able to arrange for providing the appropriate equipment to the patient for use by the patient on an on-going basis.

Prior to providing the equipment to the patient, however, medical reimbursement payor organizations such as Medicare or other private insurance companies, typically require a third party confirmation of the in-home assessment results. Of course, this involves additional scheduling and visits to the patient's home and obtaining the test equipment data. This process introduces additional delay between the physician's initial desire to provide the patient with the home therapy and the ultimate delivery of the therapeutic device or services. This third party assessment also involves additional cost to the payor organization.

Once the appropriate approvals are obtained, the home medical equipment provider arranges for the patient to receive the necessary equipment to fulfill the physician's order or prescription.

A significant shortcoming to the typical approach is that the data from the testing equipment such as an oximeter is susceptible to intentional or accidental manipulation. For example, a therapist responsible for handling the pulse oximeter and extracting the test data may not be careful enough to provide accurate results. It is possible to enter incorrect patient data, incorrect dates or other information. It is also possible with conventional arrangements to accidentally alter the data, itself, in some manner by not properly setting up the testing device to begin with, for example.

The amount of data from a typical test is significant. In an example where an individual has the pulse oximeter on their finger for a period of six hours, that may generate at least as many as 5,400 samples of pulse and oxygen level, which corresponds to at least 10,800 data points. Therefore, handling the data properly is important.

Moreover, with conventional arrangements a possibility exists for someone to manipulate pulse oximeter information in a manner to obtain payment for medical oxygen services that do not necessarily correspond to actual patient need. In some situations, there may be an intentional manipulation of data to skew the actual results such as using data from one patient more than once as if it were gathered from a plurality of patients or changing a date of test results. There are potential misuses of the data to obtain payment for oxygen services without taking the necessary steps to properly verify that the therapy is warranted. Such actions put insurance providers at risk of paying for therapy services for which the actual need has not been adequately verified. Insurance providers and payor organizations are extremely interested in obtaining proper verification but not spending large sums on measures to ensure that occurs.

There is a need for ensuring the integrity of data gathered by devices such as oximeters to avoid accidental or intentional manipulation of the data in an economical manner. This invention addresses that need.

### SUMMARY OF THE INVENTION

One of the features of the disclosed example embodiment of this invention is that it ensures the integrity of data taken from a device such as a pulse oximeter. The data is verified using at least one integrity check in a manner that greatly reduces or eliminates the possibility for accidental or intentional manipulation of the data.

An exemplary method of managing medical device data includes associating at least one security feature with the data extracted from the device. The security feature in one example includes encrypting at least some of the data. The encrypted data will readily show any manipulation of the data from its original form as provided by the oximeter or other device.

Another example includes automatically examining the content of the data or associated information and checking that against expected values to ensure that the data is correct. One disclosed example includes rejecting any data set that has any errors found as a result of comparing the data to the expected values.

An example system includes a control center that receives information from oximeters or other medical testing devices and verifies that the received data is valid. The control center has automated integrity checking capabilities.

One example system includes a communication device such as a hand-held portable device that is capable of communicating with the pulse oximeter for obtaining the pulse oximeter data. In one example, the communication device allows an authorized individual to enter information regarding a patient from whom the pulse oximeter data has been gathered. Patient information may include address, date of birth, social security number, etc.

The communication device downloads the pulse oximeter data from the pulse oximeter into the communication device. The communication device includes at least one program module that generates a file containing patient demographics and the data that is extracted from the pulse oximeter.

In another example, the oximeter and the communication device described above are integrated into a single device such that the oximeter monitoring functions and the data collection and communication functions are performed by one device. In such an example, a separate download from an oximeter to a separate communication device is not necessary.

The various features and advantages of this invention will become apparent to those skilled in the art from the following detailed description. The drawing that accompanies the detailed description can be briefly described as follows.

### BRIEF DESCRIPTION OF THE DRAWING

FIG. 1 schematically shows selected portions of a data management arrangement designed according to an embodiment of this invention.

### DETAILED DESCRIPTION

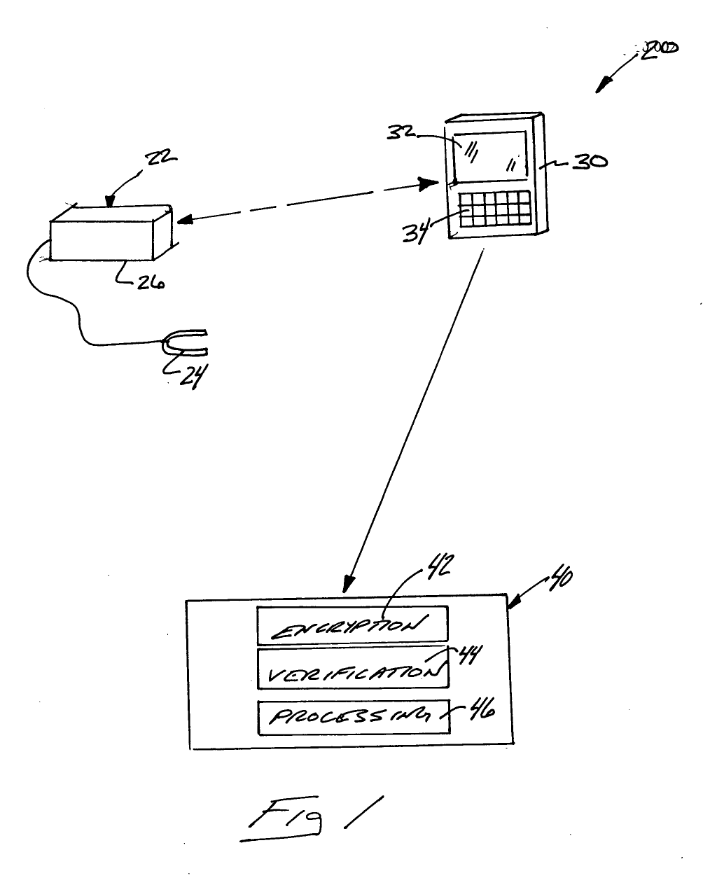

Selected portions of a data management system 20 are schematically shown in FIG. 1. A medical testing device 22, which in this example is a pulse oximeter, provides information regarding selected criteria or indicators of an individual's condition. For purposes of illustration, the testing device 22 will be referred to as a pulse oximeter in this description but this invention is not limited to any particular testing device. In this example, the oximeter 22 includes a known clip 24 that can be placed on an individual's finger for gathering information regarding pulse rate and oxygen levels. A processor portion 26 collects and stores the data points gathered by the oximeter 22.

The communication device 30 communicates with the testing device 22. In this example, the communication device 30 is a communication device 30 that can be carried about by a therapist for interacting with various testing devices such as pulse oximeters. The example communication device 30 includes a display 32 and an input interface 34 that allows an individual to enter information for creating data files, setting testing parameters, providing notifications, etc. In one example, the communication device 30 communicates with the testing device 22 using a hardwire connection. In another example, the communication device 30 communicates with the testing device 22 using known wireless communication techniques. Those skilled in the art who have the benefit of this description will be able to select appropriate techniques and components to meet the needs of their particular situation.

In this example, the communication device 30 obtains raw data from the testing device 22 and creates a data file including such data. In one example, the input interface 34 allows an authorized individual to enter information regarding a patient from whom the data has been gathered. Patient information may include their address, date of birth, social security number, etc.

At least one programming module within the communication device 30 generates a file containing the patient demographics and the data that is extracted from the testing device 22. For purposes of discussion, such a file is referred to in this description as an aggregated file.

Another portion of the example system is a control center 40 maintained at a location that is remote from the individual patient or the authorized individual using the communication device 30. The control center 40 may be facilitated through a website, for example. The control center 40 is capable of communicating with the communication device 30 so that the control center receives at least one test file from the communication device 30 that includes the data from the testing device 22 (i.e., pulse oximeter data). Once the control center 40 receives the data, it has the ability to open the data file, extract the patient demographics and analyze the recording session samples. The control center in this example generates a summarization of the test that was taken to determine whether a patient qualifies for oxygen therapy.

Protecting the data from the pulse oximeter 22 from improper or inadvertent manipulation is accomplished through the interaction between the communication device 30 and the testing device 22 on the one hand and the communication device 30 and the control center 40 on the other hand.

When the communication device 30 generates the aggregated data file containing the patient demographics and the pulse oximeter data, the data is encrypted using a secure sypher of 128 bits, in one example. The encryption ensures that the data is tamperproof. Of course, a specific effort to breach the security of the encryption would require intentional data manipulation. The encryption algorithm selected in one example is secure enough to prevent casual or accidental modification of the pulse oximeter data. Various encryption techniques may be employed to meet the needs of a particular situation and those skilled in the art who have the benefit of this description will realize what will work best for their implementation.

Once the data file from the communication device 30 is provided to the control center 40 (i.e., by being uploaded to a website), the control center performs various checks on the integrity and consistency of the data. One security technique of one example embodiment includes a cryptographic message digest algorithm that uses known SHA or MD 5 technology.

In this example, the control center 40 includes an encryption module 42 that uses appropriate cryptographic techniques for deciphering the encrypted portions of the file received from the communication device 30. It should be noted that in some examples an entire file will be encrypted. In other examples, only selected portions of the aggregate file will be encrypted. The encryption module 42 uses electronic fingerprint techniques, for example, to verify that an authorized or appropriate communication device 30 is providing the information to the control center 40.

It is worthwhile noting at this point that in one example, the communications between the communication device 30 and the control center 40 occur over a publicly available communication facility such as the internet, telephone lines or a wireless intercommunications interface. One example includes a docking station that receives the communication device 30 and facilitates a connection with the control center 40.

The encryption module 42 in another example decrypts a file received from a communication device 30 and examines the general format of the file, checking for one or more magic footprints that indicate that the file was generated by approved software. If such a checks fails the downloaded test data is not approved for processing to obtain approved payment of oxygen therapy, for example.

In one example, an electronic fingerprint, which can be called a MD 5 checksum is calculated based upon the entered file. This MD 5 checksum is stored in a database permanently in one example. Storing the MD 5 checksum provides the control center 40 with the ability to determine if the data file has already been received in the past and provides a flag for preventing using test results from a single test more than once. The MD 5 checksum in one example also provides the ability to determine when a reprint request of a previously submitted report has been made for tracking purposes.

At the control center 40, the data file is then opened and the recording session (or plurality of sessions, depending on the particular instance) is MD 5 checksummed. This provides an indication of whether the raw data from a pulse oximeter recording session has been uploaded in the past. This technique prevents inadvertent or deliberate misassignment of pulse oximeter data into a file using the communication device 30. For example, one person using a single communication device 30 may be working with more than one pulse oximeter or other testing device 22 and the MD 5 checksum procedure allows for catching an inadvertent or deliberate attempt to associate or upload a single set of data from a pulse oximeter and then assigning that data to multiple patients or the same patient for multiple dates, for example. This check allows for catching anyone intentionally or accidentally using test results from a pulse oximeter that provide an indication that oxygen therapy will be reimbursable. Ensuring that pulse oximeter data was obtained from a particular patient on a particular day is an advantageous feature of the example embodiment.

In one example, the MD 5 checksum procedure is also capable of detecting when multiple pulse oximeter test sessions are appended together in a single data file.

In one example, the data stored within the pulse oximeter is separated by each recording session. The date and time of each of the individual recording sessions is associated with the data from that session. Based upon this information, the control center stores and calculates a MD 5 checksum for each of the individual recording sessions in one example. It therefore becomes possible to detect that some or all of the sessions from a particular pulse oximeter have been previously reported. This allows for detecting intentional or inadvertent use of data from a pulse oximeter for two or more patients. It is relatively easy for an individual to inadvertently make such a mistake, for example, by forgetting to erase data from the pulse oximeter after downloading that data to the communication device 30. Such a security feature addresses a potentially common occurrence that is not associated with any wrong intent and addresses a situation that is otherwise undetectable.

The control center 40 in this example also includes a verification module 44. The example control center 40 has the ability to utilize appropriately encrypted data and to make other checks regarding the integrity of the data to guard against inadvertent or intentional misuse of data from various oximeters or other testing devices. The example verification module 44 has the ability to check at least one of several possible indicators for any received data set.

In one example, the pulse oximeter has a detailed specification that describes each bit of data that is stored in the pulse oximeter memory portion. The internal data format has start and stop dates and times for each recording session, for example. The data from the pulse oximeter in one example is stored in sets of three bytes. Two bytes of data followed by a simple checksum, which comprises the third byte. The control center analyzes the data uploaded from the communication device 30, which has been downloaded from the pulse oximeter, by checking each byte of the formatted data against the manufacturer's specification. In other words, all of the third byte checksums are recalculated and compared to the downloaded checksum. There may be other markers in the data such as an end of recording session marker and such markers are checked by the verification module 44 of the example control center 40.

In one example, every byte of data from the pulse oximeter must agree with the manufacturer's specification, otherwise the data is not allowed for processing.

Testing the data for consistency with a manufacturer's specifications includes extensive data analysis by the verification module 44 in one example. The percent saturation of oxygen in the blood can be a number from 0 to 100 percent. As a byte of data can represent up to 256 different numbers, there are 156 undefined values for a possibly valid oxygen saturation level. In one example, the verification module 44 that detects when the oxygen level is outside of a legitimate range and the entire downloaded data set for such a test is disallowed. A similar approach is useful for analyzing data regarding the values of the pulse rate.

In examples where the pulse oximeter records the start and stop time of a recording session, the verification module 44 analyzes the data to determine whether the expected number of samples were taken during that session. The verification module 44 in one example determines the number of samples that the pulse oximeter should have accumulated between the start and stop times and compares that to the actual number of samples recorded. A determined variation will result in the entire data set being disallowed.

For pulse oximeters that store date and time information as a sequence of bytes, one example verification module 44 determines whether the time information is suspect or invalid. For example, some date and time data is stored as a sequence of bytes, one for each element of time. Testing these bytes against a standard may reveal that an indication of an elapsed time of 72 minutes can be flagged as suspect or invalid even though that amount of time could have elapsed. The proper byte sequence format should indicate 1 hour and 12 minutes instead of 72 minutes. One example verification module 44 detects such a discrepancy and provides an indication that the data set is at least suspect.

Another feature of one verification module 44 is to monitor start and stop times from a particular device so that it is possible to determine whether a calendar feature of a pulse oximeter has been inaccurately set. In many instances, that information must be manually entered and there is an opportunity for mistake or intentional misrepresentation. This feature provides yet another data integrity check in examples employing this technique. A related feature in some examples is to determine whether the date and time were set, at all, by the individual responsible for setting the calendar.

One example verification module 44 has the ability to determine whether any anomalous data is appended to a test data set.

In another example, part of the manufacturer's specification includes a specific method of linking each of the recorded sessions, similar to linking a chain. The verification module 44 in one example uses old data information and a new data set link indicator to check such linking for consistency.

In one example, the verification module 44 determines how long a test session for generating the data lasted. In one example, a ten minute minimum threshold is required. Any test data that is from a session that is less than 10 minutes is considered invalid.

Another check is used regarding the patient demographics and software registration data stored in the file. If there is any inconsistency, the test data is rejected.

In examples involving three clocks, the date and time of the control center 40, the data and time in the communication device 30 and the date and time within the pulse oximeter are tested for reasonableness. This allows a verification module 44 to determine if the test administrator has been lazy, for example, and has not paid attention to details. Depending on the level of reasonableness, the test data may be disallowed. In one example, if the time on the communication device 30 recorded inside the data file is in the future, the test data is rejected. If the time in the pulse oximeter recorded at the time of the pulse oximeter download into the communication device 30 is in the future, the test data is rejected or is disallowed by the control center. If the date and time of the recorded sessions within the pulse oximeter are more than 30 days old, the test will be disallowed. In other words, this feature requires that the test administrator handle the communication device 30 with precision.

Another example security feature is to have a serial number of the communication device 30 be registered so that only authorized users of the system can provide data for analysis and processing for possible payment.

The illustrated example control center 40 includes a processing module 46 that is capable of handling a variety of data management processes. The processing module in this example is responsible for storing information that allows the verification module 44 to accomplish the various checks on data contents such as determining whether any data or data set is being potentially reused as described above, for example. The processing module 46 in this example also provides an indication whether a received data set has been approved and accepted so that the payment for medical services can proceed.

The example modules are divided up and shown schematically for discussion purposes. The described features or functions of each may be realized in one or more such modules. In one example, each module 42, 44, 46 comprises software. Given this description, those skilled in the art will realize what combination of software, hardware and firmware will work best to realize a control center that performs the functions of the disclosed example control center 40.

One example control center 40 is maintained as a website that is accessible by authorized individuals to provide data gathered from a testing device 22 and individuals authorized to analyze the results of automated data analysis to facilitate the next step in providing the prescribed therapy to an individual and to obtain payment for the necessary equipment, services or both.

The example arrangement facilitates providing oxygen therapy, for example, in the following manner. An individual gathers data from an oximeter in a secure and protected manner. That data is provided to a processing center in a secure and identified manner that protects against data manipulation or misuse. In one example, the processing center operates based on software that runs on a web server available to authorized data collectors on the internet. The processing center verifies the data and provides a validated report of the oximeter readings that is then useable for obtaining authorization, payment or both for providing the desired therapy regimen to an individual.

The preceding description is exemplary rather than limiting in nature. Variations and modifications to the disclosed examples may become apparent to those skilled in the art that do not necessarily depart from the essence of this invention. The scope of legal protection given to this invention can only be determined by studying the following claims.

We claim:

1. A method of handling data from a medical testing device, comprising:
associating at least one security feature with data from the testing device.

2. The method of claim 1, including encrypting at least a portion of the data.

3. The method of claim 1, wherein the security feature comprises at least one portion of a data file including the data from the testing device.

4. The method of claim 1, comprising certifying the data by verifying that the security feature has an expected characteristic.

5. The method of claim 1, comprising providing an indication that the data is unacceptable if there is at least one aspect of an associated security feature that differs from a corresponding expected aspect.
